(19) United States
(12) Patent Application Publication
(10) Pub. No.: US 2025/0144426 A1
(43) Pub. Date: May 8, 2025

(54) Treating Conditions Impacted by the Splanchnic Bed Using Spinal Cord Stimulation

(71) Applicant: Boston Scientific Neuromodulation Corporation, Valencia, CA (US)

(72) Inventor: Ismael Huertas Fernandez, Seville (ES)

(21) Appl. No.: 18/931,887

(22) Filed: Oct. 30, 2024

Related U.S. Application Data
(60) Provisional application No. 63/595,616, filed on Nov. 2, 2023.

(51) Int. Cl. A61N 1/36; A61N 1/05; A61N 1/372

(52) U.S. Cl. CPC A61N 1/36139; A61N 1/0551; A61N 1/36062; A61N 1/37217; A61N 1/37247

(57) ABSTRACT

Techniques are disclosed using a Spinal Cord Stimulation (SCS) Implantable Pulse Generator (IPG) to treat conditions impacted by the splanchnic bed. For example, heart failure can be treated by increasing the recruitment of blood volume to the organs in a patient's splanchnic bed, thus alleviating stress on the heart. When employed in this context, non-destructive stimulation is preferably provided to the dorsal horn in the patient's spinal column, which modulates the intermediolateral nucleus (IML) that includes sympathetic pre-ganglionic neurons (SPNs) that ultimately affect the sympathetic nervous system of splanchnic nerves innervating the splanchnic bed. Use of the IPG can be supplemented by use of a sensor, such as a peripheral blood pressure sensor, which can be used to assess the effectiveness of the stimulation in modulating the recruitment of blood volume in the splanchnic bed, and to provide closed loop control of the IPG.

### CROSS REFERENCE TO RELATED APPLICATIONS

This application is a non-provisional of U.S. Provisional Patent Application Ser. No. 63/595,616, filed Nov. 2, 2023, which is incorporated herein by reference in its entirety, and to which priority is claimed.

### FIELD OF THE INVENTION

This application relates to Implantable Spinal Cord Stimulator (SCS) devices, and more specifically to use of such devices in treating conditions impacted by the splanchnic bed.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system. However, the present invention may find applicability with any stimulator device system.

A stimulator system typically includes an Implantable Pulse Generator (IPG) 10 shown in FIG. 1. The IPG 10 includes a biocompatible device case 12 that holds the circuitry and a battery 14 for providing power for the IPG to function. The IPG 10 is coupled to tissue-stimulating electrodes 16 via one or more electrode leads 15 that form an electrode array 17. For example, one or more percutaneous leads 15 can be used having ring-shaped or split-ring electrodes 16 carried on a flexible body 18. In another example not illustrated, a paddle lead can provide an electrode array 17 positioned on one of its generally flat surfaces. Lead wires 20 within the leads are coupled to the electrodes 16 and to proximal contacts 22 insertable into lead connectors 24 fixed in a header 23 on the IPG 10, which header can comprise an epoxy for example. Once inserted, the proximal contacts 22 connect to header contacts within the lead connectors 24, which are in turn coupled by feedthrough pins to stimulation circuitry 28 (FIG. 3) within the case 12.

In the illustrated IPG 10, there are sixteen electrodes (E1-E16), split between two percutaneous leads 15, and thus the header 23 may include two lead connectors 24. However, the type and number of leads, and the number of electrodes, and the number of lead connectors in an IPG is application specific and therefore can vary. The conductive case 12, or some conductive portion of the case, can also comprise an electrode (Ec). In an SCS application, the electrode lead(s) 15 are typically implanted in the spinal column inside the patient's vertebrae and proximate to the dura in a patient's spinal cord, preferably spanning left and right of the patient's spinal column. The proximal contacts 22 are tunneled through the patient's tissue to a distant location such as the buttocks where the IPG case 12 is implanted, at which point they are coupled to the lead connectors 24. SCS therapy is traditionally used to relieve symptoms such as chronic back pain. IPG 10 as described should be understood as including non-implantable External Trial Stimulators (ETSs), which mimic operation of the IPG 10 during trials periods when leads have been implanted in the patient but the IPG 10 has not. See, e.g., U.S. Pat. No. 9,259,574 (disclosing an ETS).

IPG 10 can include an antenna 26a allowing it to communicate bi-directionally with a number of external devices discussed subsequently. Antenna 26a as shown comprises a conductive coil within the case 12, although this coil antenna can also appear in the header 23. When antenna 26a is configured as a coil, communication with external devices preferably occurs using near-field magnetic induction. IPG 10 may also include a Radio-Frequency (RF) antenna 26b. In FIG. 1, RF antenna 26b is shown within the header 23, but it may also be within the case 12. RF antenna 26b may comprise a patch, slot, or wire, and may operate as a monopole or dipole. RF antenna 26b preferably communicates using far-field electromagnetic waves, and may operate in accordance with any number of known RF communication standards, such as Bluetooth, Zigbee, WiFi, MICS, and the like.

Stimulation in IPG 10 is typically provided by pulses each of which may include a number of phases (30), as shown in the example of FIG. 2. Stimulation parameters typically include amplitude (current I, although a voltage amplitude V can also be used); frequency (F); pulse width (PW); the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2, electrode E1 has been selected as an anode (during its first phase 30a), and thus provides pulses which source a positive current of amplitude +I to the tissue. Electrode E2 has been concurrently selected as a cathode (again during first phase 30a), and thus provides pulses which sink a corresponding negative current of amplitude-I from the tissue. This is an example of bipolar stimulation, in which the electrode array 17 includes one anode pole and one cathode pole, as discussed later with respect to FIG. 5A. Stimulation provided by the IPG 10 can also be monopolar, with the electrode array 17 programmed with a single pole of a given polarity (e.g., a cathode pole), and with the conductive case electrode Ec acting as a return (e.g., an anode pole). Multipolar (e.g., tripolar) stimulation can also be used, with the electrode array 17 having three or more poles. Note that more than one electrode in the electrode array may be active to form a pole in the electrode array, as discussed and shown further below. See also U.S. Pat. No. 10,881,859, which is incorporated herein by reference in its entirety.

IPG 10 as mentioned includes stimulation circuitry 28 to form prescribed stimulation at a patient's tissue, and FIG. 3 shows an example of such circuitry. The stimulation circuitry 28 shown includes one or more current source circuits and one or more current sink circuits. The sources and sinks can comprise Digital-to-Analog converters (DACs), and may be referred to as PDACs and NDACs in accordance with the Positive (sourced, anodic) and Negative (sunk, cathodic) currents they respectively issue. In the example shown, a NDACi/PDACi pair is dedicated (hardwired) to a particular electrode node ei 39. Each electrode node ei 39 is associated with an electrode Ei 16 via a DC-blocking capacitor Ci 38, for the reasons explained below. The stimulation circuitry 28 in this example also supports selection of the conductive case 12 as an electrode (Ec 12), which case electrode is typically selected for monopolar stimulation as explained above. PDACs and NDACs can also comprise voltage sources.

Proper control of the PDACs and NDACs allows any of the electrodes 16 to act as anodes or cathodes to create a current through a patient's tissue, R, hopefully with good therapeutic effect. Consistent with the example provided in FIG. 2, FIG. 3 shows operation during the first phase 30a in which electrode E1 has been selected as an anode electrode to source current I to the tissue R and E2 has been selected as a cathode electrode to sink current I from the tissue. Thus PDAC1 and NDAC2 are digitally programmed (Ip1, In2) to produce the desired current, I, with the correct timing (e.g., in accordance with the prescribed frequency and pulse widths). As mentioned above, more than one anode electrode and more than one cathode electrode may be selected at one time, and thus current can flow through the tissue R between two or more of the electrodes 16. Other stimulation circuitries 28 can also be used in the IPG 10, including ones that include switching matrices between the electrode nodes ei 39 and the N/PDACs. See, e.g., U.S. Pat. Nos. 6,181,969, 8,606,362, 8,620,436, 11,040,192, and 10,912,942. Much of the stimulation circuitry 28 of FIG. 3, including the PDACs and NDACs, the switch matrices (if present), and the electrode nodes ei 39 can be integrated on one or more Application Specific Integrated Circuits (ASICs), as described in U.S. Patent Application Publications 2012/0095529, 2012/0092031, and 2012/0095519. As explained in these references, ASIC(s) may also contain, or be coupled with, other circuitry useful in the IPG 10, such as a microcontroller, telemetry circuitry (for interfacing off chip with telemetry antennas 26a and/or 26b), circuitry for generating the compliance voltage VH which powers the stimulation circuitry 28, various measurement circuits, etc. Collectively, such circuitry comprises control circuitry in the IPG 10.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861. While useful, DC-blocking capacitors 38 are not strictly required in all IPG designs and applications.

Referring again to FIG. 2, the stimulation pulses as shown are biphasic, with each pulse comprising a first phase 30a followed thereafter by a second phase 30b of opposite polarity. Biphasic pulses are useful to actively recover any charge that might be stored on capacitive elements in the electrode current paths, such as on the DC-blocking capacitors 38. During the first pulse phase 30a (e.g., PDAC1 and NDAC2 are activated), charge will (primarily) build up across the DC-blockings capacitors (e.g., C1 and C2) associated with the electrodes (e.g., E1 and E2) used to produce the current. During the second pulse phase 30b, when the polarity of the current I is reversed at the selected electrodes El and E2 (e.g., PDAC2 and NDAC1 are activated), the stored charge on capacitors C1 and C2 is recovered.

Charge recovery using phases 30a and 30b is said to be “active” because the P/NDACs in stimulation circuitry 28 actively drive a current, in particular during second phase 30b to recover charge stored after the first phase 30a. However, such active charge recovery may not be perfect, and some residual charge may be present in capacitive elements in the current path even after phase 30b is completed. Accordingly, the stimulation circuitry 28 can also provide for passive charge recovery. Passive charge recovery is implemented using passive charge recovery switches 41 as shown in FIG. 3. These switches 41 when selected via assertion of control signals Xi couple each electrode node ei to a particular circuit node (shown here as the battery voltage Vbat, although another DC node could be used as well). As explained in U.S. Pat. Nos. 10,716,937 and 10,792,491, this allows any stored charge to be recovered through the patient's tissue, R. Control signals Xi are usually asserted to cause passive charge recovery after each pulse (e.g., after phase 30b) during periods 30c shown in FIG. 2, and are at least asserted in the previously active current paths: that is, at least X1 and X2 would be asserted in the example of FIG. 2 (although all control signals Xi could also be asserted). Because passive charge recovery involves capacitive discharge through the resistance R of the patient's tissue, such discharge manifests as an exponential decay in current, although this is not shown in FIG. 2. Passive charge recovery during period 30c may be followed by a quiet period 30d during which no active current is driven by the DAC circuitry, and none of the passive recovery switches 41 are closed. This quiet period 30d may last until the next pulse is actively produced (e.g., phase 30a). Like the particulars of pulse phases 30a and 30b, the occurrence of passive charge recovery (30c) and any quiet periods (30d) can be prescribed as part of the stimulation program.

Although not shown in FIG. 2, stimulation pulses can also be monophasic, having only a single actively driven phase (30a). Because monophasic pulses lack an active charge recovery phase (30b), such monophasic pulses would typically be followed by passive charge recovery (30c) as just described.

FIG. 4 shows various external systems 60, 70, and 80 that can wirelessly communicate data with the IPG 10. Such systems can be used to wirelessly transmit a stimulation program to the IPG 10—that is, to program its stimulation circuitry 28 to produce stimulation with desired amplitudes and timings as described earlier. Such systems may also be used to adjust one or more stimulation parameters of a stimulation program that the IPG 10 is currently executing, and/or to wirelessly receive information from the IPG 10, such as various status information, etc.

External controller 60 can be as described in U.S. Patent Application Publication 2015/0080982 for example, and may comprise a portable, hand-held controller dedicated to work with the IPG 10. External controller 60 may also comprise a general-purpose mobile electronics device such as a mobile phone which has been programmed with a Medical Device Application (MDA) allowing it to work as a wireless controller for the IPG 10, as described in U.S. Patent Application Publication 2015/0231402. External controller 60 includes a display 61 and a means for entering commands, such as buttons 62 or selectable graphical icons provided on the display 61. The external controller 60's user interface enables a patient to adjust stimulation parameters, although it may have limited functionality when compared to systems 70 and 80, described shortly. The external controller 60 can have one or more antennas capable of communicating with the IPG 10. For example, the external controller 60 can have a near-field magnetic-induction coil antenna 64a capable of wirelessly communicating with the coil antenna 26a in the IPG 10. The external controller 60 can also have a far-field RF antenna 64b capable of wirelessly communicating with the RF antenna 26b in the IPG 10.

Clinician programmer 70 is described further in U.S. Patent Application Publication 2015/0360038, and can comprise a computing device such as a desktop, laptop, or notebook computer, a tablet, a mobile smart phone, a Personal Data Assistant (PDA)-type mobile computing device, etc. In FIG. 4, the computing device is shown as a laptop computer that includes typical computer user interface means such as a display 71, buttons 72, as well as other user-interface devices such as a mouse, a keyboard, speakers, a stylus, a printer, etc., not all of which are shown for convenience. Also shown in FIG. 4 are accessory devices for the clinician programmer 70 that are usually specific to its operation as a stimulation controller, such as a communication “wand” 76 coupleable to suitable ports on the computing device. The antenna used in the clinician programmer 70 to communicate with the IPG 10 can depend on the type of antennas included in the IPG 10. If the patient's IPG 10 includes a coil antenna 26a, wand 76 can likewise include a coil antenna 74a to establish near-field magnetic-induction communications at small distances. In this instance, the wand 76 may be affixed in close proximity to the patient, such as by placing the wand 76 in a belt or holster wearable by the patient and proximate to the patient's IPG 10. If the IPG 10 includes an RF antenna 26b, the wand 76, the computing device, or both, can likewise include an RF antenna 74b to establish communication with the IPG 10 at larger distances. The clinician programmer 70 can also communicate with other devices and networks, such as the Internet, either wirelessly or via a wired link provided at an Ethernet or network port.

External system 80 comprises another means of communicating with and controlling the IPG 10 via a network 85 which can include the Internet. The network 85 can include a server 86 programmed with communication and control functionality, and may include other communication networks or links such as WiFi, cellular or land-line phone links, etc. The network 85 ultimately connects to an intermediary device 82 having antennas suitable for communication with the IPG's antenna, such as a near-field magnetic-induction coil antenna 84a and/or a far-field RF antenna 84b. Intermediary device 82 may be located generally proximate to the IPG 10. Network 85 can be accessed by any user terminal 87, which typically comprises a computer device associated with a display 88. External system 80 allows a remote user at terminal 87 to communicate with and control the IPG 10 via the intermediary device 82.

FIG. 4 also shows circuitry 90 involved in any of external systems 60, 70, or 80. Such circuitry can include control circuitry 92, which can comprise any number of devices such as one or more microprocessors, microcomputers, FPGAs, DSPs, other digital logic structures, etc., which are capable of executing programs in a computing device. Such control circuitry 92 may contain or coupled with memory 94 which can store external system software 96 for controlling and communicating with the IPG 10, and for rendering a Graphical User Interface (GUI) 99 on a display (61, 71, 88) associated with the external system. In external system 80, the external system software 96 would likely reside in the server 86, while the control circuitry 92 could be present in either or both the server 86 or the terminal 87.

FIG. 5A shows GUI 99 as may be rendered on an external system to program the stimulation the IPG 10 provides. The GUI 99 includes a waveform interface 100 which allows certain stimulation parameters (amplitude I, pulse width PW, and frequency F) to be set or adjusted. Although not shown, waveform interface 100 can include options to set other parameters for the stimulation waveform, like whether biphasic or monophasic pulses are used, whether active and/or passive recovery is to be used, etc. GUI 99 also includes an electrode configuration interface 102 which allows for the selection of electrodes 16 in the electrode array 17 that will provide the stimulation. Interface 102 as shown allows a user to select whether an electrode will operate as an anode, a cathode, or be off (inactive). Further, the percentage of the amplitude (X %) at each active electrode can be specified. In the example shown, electrodes E2, E12, and E13 have been selected to act as anodes, with these electrodes receiving 70, 15, and 15% of the amplitude I respectively as an anodic current. That is, E2 will provide 0.7*+I, while E12 and E13 each provide 0.15*+I. Electrodes E3, E4, and E14 have been selected to act as cathodes, with these electrodes receiving 40, 40, and 20% of the amplitude I respectively as a cathodic current. That is, E3 and E4 will each provide 0.4*-I, while E14 will provide 0.2*-I. Examples of these waveforms and their relative amplitudes are shown in FIG. 5B, and are shown using biphasic pulses with a first phase 30a having the polarity specified in interface 102 and a second phase 30b of opposite polarity.

GUI 99 in this example also includes a visualization interface 104. Preferably, this interface 104 shows the positioning of leads 15 in the electrode array 17 relative to each other as they are implanted in the patient, and relative to certain tissue structures in the patient such as various vertebrae Vi. These vertebrae could be cervical (C), thoracic (T) lumbar (L), or sacral (S) vertebrae depending where the leads 15 have been implanted in the patient. Other relevant tissue structures could be shown in interface 104 as well. The tissue structures as shown in visualization interface 104 preferably comes from imaging information (e.g., fluoroscopy) taken from the patient.

The visualization interface 104 can also preferably show some indication of the stimulation being provided that is overlaid over the tissue structure and the lead(s) 15. For example, different shading can be used to show which electrodes have been selected to act as anodes (dark), cathodes (light), or that are off (grey). Furthermore, a position of the poles formed by the active electrodes can also be shown. For example, because electrodes E2, E12 and E13 act as anodes, they establish an anode pole (+) at a position in the electrode array 17 influenced by the magnitudes of the anodic current provided at these electrodes (i.e., in between E12 and E13, but closest to E2 because that electrode provides the largest anodic current). Similarly, because electrodes E3, E4 and E14 act as cathodes, they establish a cathode pole (−) at a position influenced by the magnitudes of the cathodic current provided at these electrodes (i.e., in between E3 and E4, and closer to these electrodes because they provide larger cathodic currents). As this example shows, and as mentioned earlier, a pole can be formed in the electrode array 17 using one or more active electrodes (here, three electrodes are used to make each of the anode pole and the cathode pole). This example also illustrates bipolar stimulation, which involves use of a single anode (+) and cathode (−) pole in the electrode array 17. As mentioned earlier, however, stimulation can also be monopolar or multipolar.

As discussed further in U.S. Pat. No. 10,881,859, an electrode configuration algorithm operable in or with the external system rendering GUI 99 can be used to determine the position of the poles given the selection of the electrodes in the electrode configuration interface 102, and as such can indicate the positions of these poles in the visualization interface 104. This algorithm can also operate in reverse. For example, a user can position the anode and/or cathode poles in the electrode array 17 in the visualization interface 104 (using a mouse cursor for example), with the electrode configuration algorithm then operating in reverse to determine which electrodes should be active and with which polarities and amplitudes in electrode configuration interface 102, to form the poles at the specified positions.

### SUMMARY

A method for treating at least one condition impacted by a patient's splanchnic bed using spinal cord stimulation. The method may comprise: providing an electrode array having a plurality of electrodes within a patient's spinal column; and providing from a spinal cord stimulator non-destructive electrical stimulation at at least one of the plurality of the electrodes, wherein the stimulation is configured to affect the sympathetic nervous system innervating the patient's splanchnic bed to modulate one or more of (i) activity of one or more organs in the splanchnic bed, and (ii) a blood volume of the splanchnic bed.

In one example, the stimulation is configured to increase the blood volume of the splanchnic bed. In one example, the stimulation stimulates one or more dorsal horns of the patient's spinal cord. In one example, the electrode array is provided longitudinally within the spinal column to configure the stimulation to affect at least one splanchnic nerve innervating the patient's splanchnic bed. In one example, the stimulation comprises a bipole in the electrode array. In one example, the stimulation comprises a monopole in the electrode array. In one example, the stimulation is not perceptible by the patient. In one example, the stimulation is provided using at least one first pole during first durations and at least one second pole during second durations, wherein the first and second durations are interleaved in time. In one example, the electrode array comprises a plurality of electrode leads. In one example, a first of the electrode leads is positioned proximate to a first dorsal horn of the patient, and wherein a second of the electrode leads is positioned proximate to a second dorsal horn of the patient. In one example, the method further comprises measuring a parameter indicative of at least one of the conditions impacted by the patient's splanchnic bed. In one example, the parameter is objectively measured. In one example, the parameter is subjectively measured. In one example, the parameter comprises a cardiovascular parameter indicative of a cardiovascular status of the patient. In one example, the cardiovascular parameter is indicative of the blood volume of the patient's splanchnic bed. In one example, the parameter is measured by a sensor. In one example, the parameter comprises a blood pressure of the patient. In one example, the blood pressure is measured outside of the patient's splanchnic bed. In one example, the method further comprises adjusting at the spinal cord stimulator the stimulation using the measured parameter. In one example, the parameter is measured periodically, and wherein the stimulation is adjusted in a closed loop fashion using the periodically measured parameter. In one example, the method further comprises wirelessly receiving the measured parameter at the spinal cord stimulator. In one example, the measured parameter is input using a graphical user interface of an external system in communication with the spinal cord stimulator. In one example, the method further comprises wirelessly receiving the measured parameter at an external system in communication with the spinal cord stimulator. In one example, the parameter is further displayed on a display of the external system.

A system is disclosed for treating at least one condition impacted by a patient's splanchnic bed using a spinal cord stimulator comprising an electrode array having a plurality of electrodes configured to be provided in a patient's spinal column. The system may comprise: control circuitry configured to cause non-destructive electrical stimulation at at least one of the plurality of the electrodes, wherein the stimulation is configured to affect the sympathetic nervous system innervating the patient's splanchnic bed to modulate one or more of (i) activity of one or more organs in the splanchnic bed, and (ii) a blood volume of the splanchnic bed; wherein the control circuitry is further configured to receive a parameter indicative of at least one of the conditions impacted by the patient's splanchnic bed.

In one example, the stimulation is configured to increase the blood volume of the splanchnic bed. In one example, the system further comprises the spinal cord stimulator, wherein the control circuitry is within the spinal cord stimulator. In one example, the electrode array is configured to stimulate one or more dorsal horns of the patient's spinal cord. In one example, the electrode array is configured to cause the stimulation to affect at least one splanchnic nerve innervating the patient's splanchnic bed. In one example, the control circuitry is configured to wirelessly receive the parameter. In one example, the electrode array comprises a plurality of electrode leads. In one example, a first of the electrode leads is configured to be positioned proximate to a first dorsal horn of the patient, and wherein a second of the electrode leads is configured to be positioned proximate to a second dorsal horn of the patient. In one example, the stimulation is configured as a bipole in the electrode array. In one example, the stimulation is configured as a monopole in the electrode array. In one example, the stimulation is configured to be not perceptible by the patient. In one example, the stimulation is configured as at least one first pole during first durations and at least one second pole during second durations, wherein the first and second durations are interleaved in time. In one example, the parameter is objectively measured. In one example, the parameter is subjectively measured. In one example, the parameter comprises a cardiovascular parameter indicative of a cardiovascular status of the patient. In one example, the cardiovascular parameter is indicative of the blood volume of the patient's splanchnic bed. In one example, the system further comprises a sensor configured to measure the parameter. In one example, the parameter comprises a blood pressure of the patient. In one example, the sensor is configured to measure the parameter outside of the patient's splanchnic bed. In one example, the control circuitry is further configured to adjust the stimulation using the parameter. In one example, the parameter is received periodically, and wherein the control circuitry is configured to adjust the stimulation in a closed loop fashion using the periodically received parameter. In one example, the system further comprises an external system configured for communication with the spinal cord stimulator, wherein the control circuitry is within the external system. In one example, the parameter is input using a graphical user interface of the external system. In one example, the external system is configured to wirelessly receive the parameter. In one example, the parameter is further displayed on a display of the external system.

### BRIEF DESCRIPTION OF THE DRAWINGS

FIG. 1 shows an Implantable Pulse Generator (IPG), in accordance with the prior art.

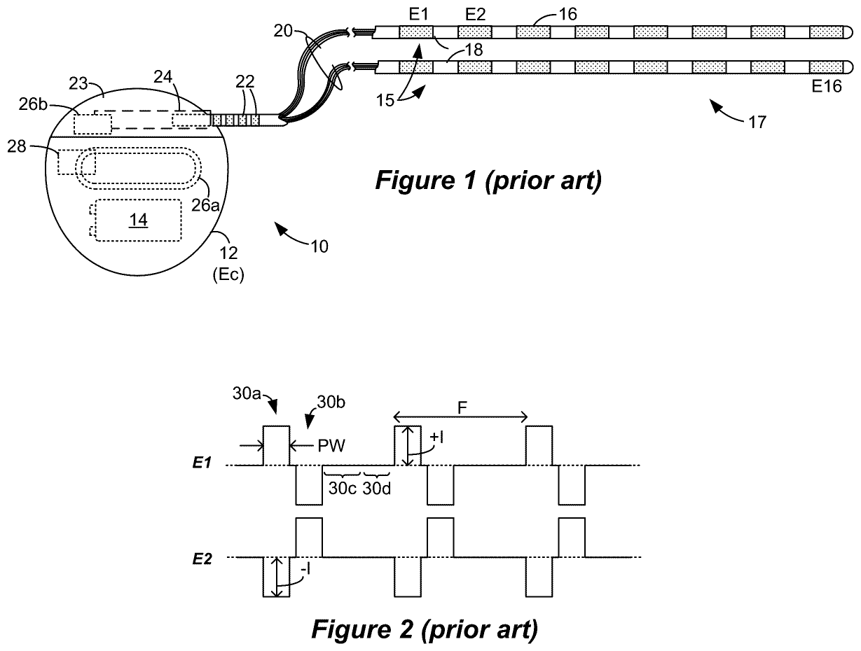

FIG. 2 shows an example of stimulation pulses producible by the IPG, in accordance with the prior art.

FIG. 3 shows stimulation circuitry useable in the IPG, in accordance with the prior art.

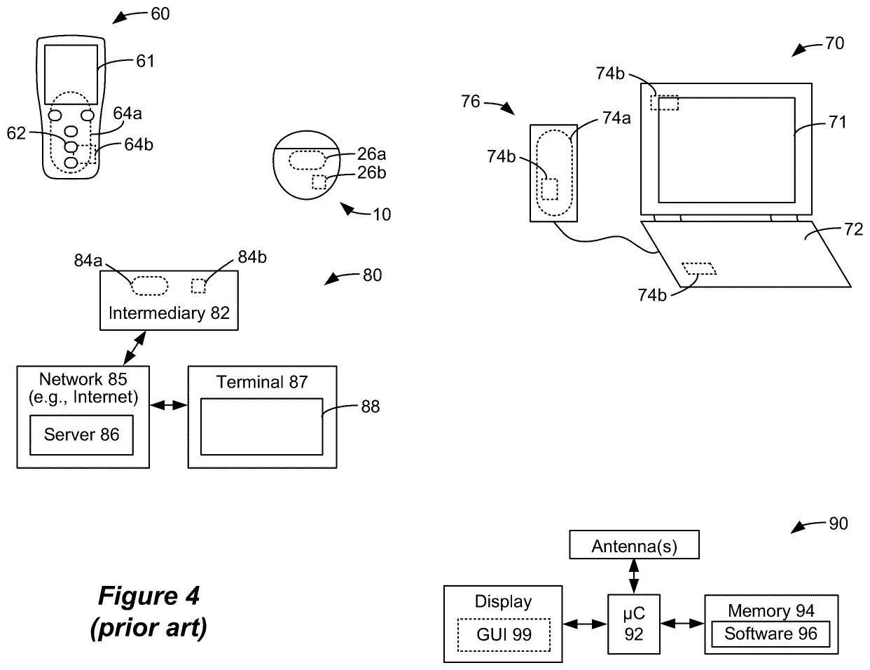

FIG. 4 shows various external devices capable of communicating with and programming stimulation in an IPG, in accordance with the prior art.

FIG. 5A shows a GUI present on an external system to program simulation in an IPG, with FIG. 5B showing an example of waveforms specified by the GUI, in accordance with the prior art.

FIG. 6 shows relevant physiological structures of the spinal cord in the spinal column, and the positioning of the leads relative to these structures in the spinal column, as useful to treating conditions impacted by the splanchnic bed using spinal cord stimulation.

FIG. 7 shows the GUI on an external system used to program simulation in a SCS IPG, and shows preferable stimulation parameters that can be used in treating conditions impacted by the splanchnic bed.

FIG. 8 shows system elements that can be involved in controlling the SCS IPG, including a sensor capable of wirelessly communicating with an external system and/or the IPG.

FIG. 9 shows an algorithm for closed loop control of SCS stimulation to treat conditions impacted by the splanchnic bed using the sensor or subjective measurements.

### DETAILED DESCRIPTION

As discussed earlier, Spinal Cord Stimulation (SCS) is typically used to treat symptoms such as back pain. However, the inventor sees other uses for SCS, in particular to treat conditions impacted by the splanchnic bed of a patient. The splanchnic bed comprises certain organs in the abdomen, such as the liver, stomach, spleen, pancreas, and intestines.

It has been theorized that at least some forms of heart failure or other cardiovascular conditions may result from an improper allotment of blood capacity in the human body. In heart failure patients, the sympathetic nervous system can be overactive. This sends signals via various splanchnic nerves to the splanchnic bed. Together, organs in the splanchnic bed typically hold up to 50% of the body's blood volume. When the sympathetic nervous system is overactive, the splanchnic nerves cause blood vessels in the splanchnic bed to constrict. As a result, the splanchnic bed cannot hold as much blood, meaning more peripheral structures in the body, such as the heart and lungs, will need to carry an excessive blood capacity. This creates higher blood pressures, and places additional stress on the heart and peripheral cardiovascular structures, leading to, or exacerbating, heart failure or other cardiovascular conditions.

Improper metabolic activity of organs in the splanchnic bed can cause other troublesome conditions. For example, improper insulin release by the pancreas or lipid metabolism by the liver can cause improper blood levels of for example glucose, triglycerides, low-and high-density lipoproteins (LDL, HDL), and cholesterol, which play a key role in diabetes and cardiovascular conditions.

Prior art techniques to treat the sympathetic nervous system involve ablation of relevant splanchnic nerves. For example, it has been taught to ablate the right greater splanchnic nerve. This is accomplished using a small RF catheter inserted through the groin.

While this prior art technique is minimally invasive and does not require a permanent implant, it suffers from the drawback of being a destructive technique, as it requires the destruction of the patient's nerves. As such, this prior art technique is permanent and not reversible. Furthermore, this prior art technique is not modulatable: it requires complete destruction of the patient's nerves, even though these nerves (even if not acting perfectly) may still be somewhat functional.

The inventor proposes using non-destructive Spinal Cord Stimulation (SCS) to treat conditions impacted by the splanchnic bed. Treating such conditions may be affected by modulating the activity of the sympathetic nervous system and the splanchnic nerves to in turn modulate activity of: (i) one or more organs in the splanchnic bed, or (ii) a blood volume of the splanchnic bed, or both. Evidence suggests that such treatment will treat a number of conditions such as heart failure and other cardiovascular conditions such as hypertension, arteriosclerosis, coronary and peripheral artery disease, dyslipidemia, hypercholesterolemia, diabetes, and other conditions that are affect by poor regulation of the organs in the splanchnic bed.

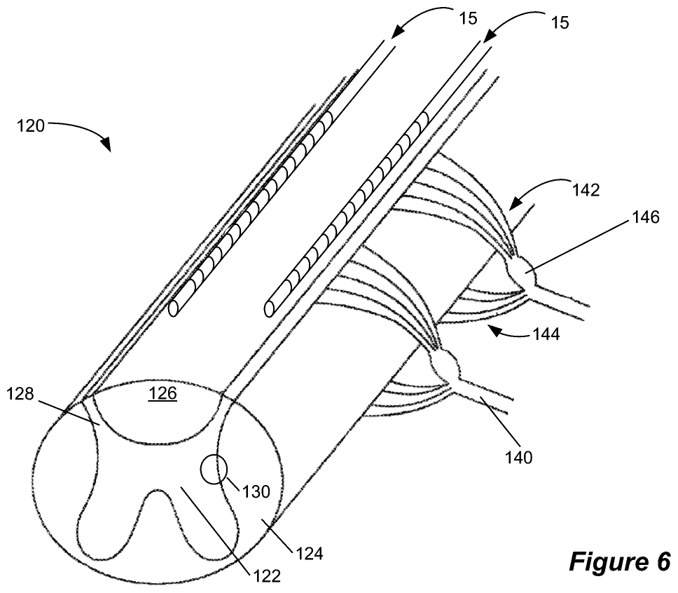

FIG. 6 shows the physiology of the spinal cord 120 within the spinal column, with vertebrae surrounding the spinal cord removed for convenience. A typical transverse section of the spinal cord 120 includes a central “butterfly” shaped central area of grey matter 122 substantially surrounded by an elliptical outer area of white matter 124. The white matter 124 of the dorsal column (DC) 126 includes mostly large myelinated axons that form afferent fibers that run in an longitudinal (rostral/caudal) direction. The dorsal portions of grey matter 122 are referred to as dorsal horns (DH) 128. In contrast to the DC fibers that run in an longitudinal direction, DH fibers can be oriented in many directions, including laterally with respect to the longitudinal axis of the spinal cord. Also shown is the general position of the intermediolateral nucleus (IML) 130 in the grey natter 122. The IML 130 includes sympathetic pre-ganglionic neurons (SPNs) which ultimate innervate various splanchnic nerves as discussed further below.

Also shown in FIG. 6 are spinal nerves 140 that are connected to the spinal cord 120. Spinal nerves 140 are split into a dorsal root (DR) 142 and a ventral root 144, each of which comprise subdivisions referred to as rootlets. The dorsal root 142 also includes a structure called the dorsal root ganglion (DRG) 146, which comprises cell bodies of the afferent neurons. The dorsal roots 142 contains afferent neurons, meaning that they carry sensory signals into the spinal cord, while the ventral roots 714 functions as an efferent motor roots.

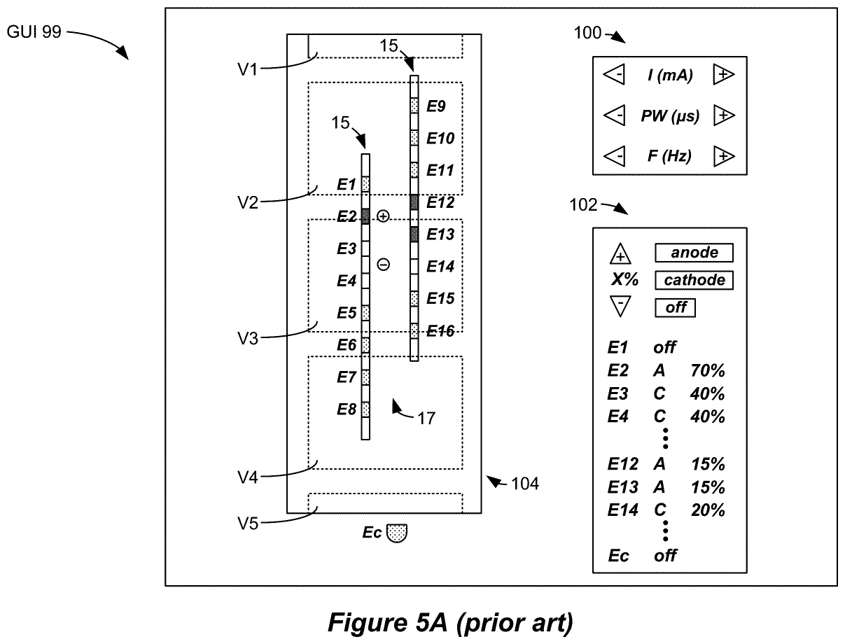

When using spinal cord stimulation to treat conditions impacted by the splanchnic bed, it is desirable to non-destructively electrically stimulate the IML 130 (or other grey matter structures coupled to it, like the dorsal horns 128) proximate to spinal nerves 140 that connect to the various splanchnic nerves that innervate the splanchnic bed. As already mentioned, the IML 130 comprises sympathetic pre-ganglionic neurons (SPNs), the modulation of which will modulate operation of one or more the splanchnic nerves. Such splanchnic nerves can include the greater splanchnic nerve connected to spinal nerves 140 proximate to thoracic vertebrae T5-T9; the lesser splanchnic nerve connected to spinal nerves 140 proximate to thoracic vertebrae T10-T11; the least splanchnic nerve connected to spinal nerves 140 proximate to thoracic vertebra T12; and/or the lumbar or sacral splanchnic nerves connected to spinal nerves 140 proximate to lumbar vertebrae L1-L2. To modulate one or more of these splanchnic nerves, the leads 15 in the electrode array 17 should be properly positioned relative to the spinal cord, both longitudinally (i.e., at the correct vertebral level for the splanchnic nerve in question) and laterally such that the leads 15 are proximate to the left and right dorsal horns 128 as shown in FIG. 6. When treating conditions impacted by the splanchnic bed, spinal cord stimulation is preferably applied at thoracic levels T6-T12, which modulates activity on the greater, lesser, and least splanchnic nerves. programming useful for treating conditions impacted by the splanchnic bed. GUI 99 is shown as implemented on an external system, and in particular is as might be present on the clinician programmer 70 (FIG. 4). As such, GUI 99 can be useful to the clinician when fitting the patient, i.e., when setting or adjusting stimulation parameters to best treat a patient's symptoms. However, GUI 99, or portions thereof, may also be present on the patient's external controller 60 (FIG. 4), where it can likewise be used to set or adjust stimulation.

As shown in the visualization interface 104, the leads 15 in the electrode array 17 are positioned in the spinal column proximate to the T9-T12 vertebrae, which as noted above, are generally proximate to spinal nerves that couple to the greater, lesser, and least splanchnic nerves. The inventor expects that positioning of the leads 15 at these locations would provide the best opportunity to modulate the sympathetic nervous system affecting the splanchnic bed (e.g., at lower thoracic positions). That being said, the leads 15 could be positioned elsewhere, i.e., proximate to spinal nerves coupled to splanchnic nerves at other longitudinal positions. As noted earlier, the leads 15 are preferably positioned close to the dorsal horns 128 at these longitudinal locations, due to the dorsal horn 128's connection to the IML 130 that affects the sympathetic nervous system.

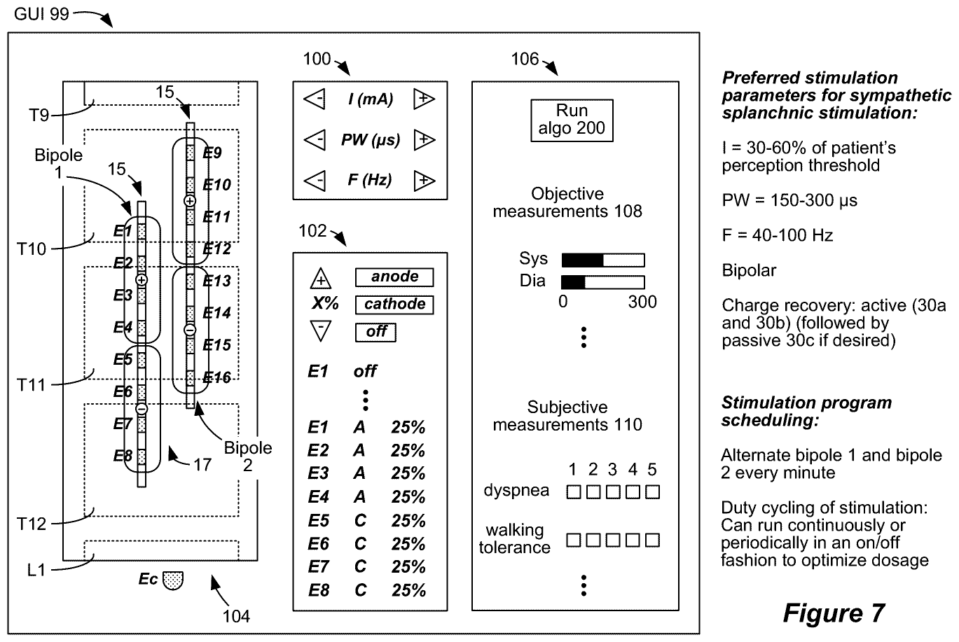

When spinal cord stimulation is used to treat pain, it is typically advisable to provide stimulation via an electric field at a concentrated position in the electrode array 17. This is typically provided by bipolar stimulation with an anode and cathode pole in the electrode array 17 that are close together, and at a location that precisely recruits the patient's pain. However, when treating conditions impacted by the splanchnic bed, the inventor hypothesizes that a more diffuse electric field can be used that is less targeted to a particular position. Therefore, as shown in FIG. 7, SCS stimulation in this context can employ a bipole that is more diffused and spread in the electrode array. For example, a first bipole (bipole 1) can be formed using all of the electrodes on one of the leads 15, with four being used to form the anode pole (E1-E4, each providing 25% of the anodic current, 0.25*+I), and four being to form the cathode pole (E5-E8, each providing 25% of the cathodic current, 0.25*-I), as shown in the electrode configuration interface 102. Examples of how bipole 1 may be formed and configured are disclosed in U.S. Pat. Nos. 10,549,097 and 11,376,433, and U.S. Patent Application Publication 2022/0296902 .

Furthermore, when used to treat conditions impacted by the splanchnic bed, it is preferable that the stimulation provided is not perceptible to the patient. This is in contrast to the use of SCS to treat pain, because in that context the perceived stimulation (paresthesia) can be useful to “cover” the pain that the patient is feeling. However, when using SCS stimulation to treat conditions impacted by the splanchnic bed and modulate the sympathetic nerves innervating the organs of the splanchnic bed, it is not necessary that the patient feel the spinal cord stimulation and experience paresthesia. As such, it is preferable in this context that the stimulation be below the patient's perception threshold. This can occur as follows. Once appropriate stimulation is determined for the patient (e.g., frequency, pulse width, and active electrodes have been set), the amplitude I of the stimulation can be adjusted to be below that which the patient can feel. As noted in FIG. 7, this sub-perception level can be 30 to 60% of the patient's perception threshold—i.e., 30 to 60% of the amplitude I required for the patient to feel the stimulation.

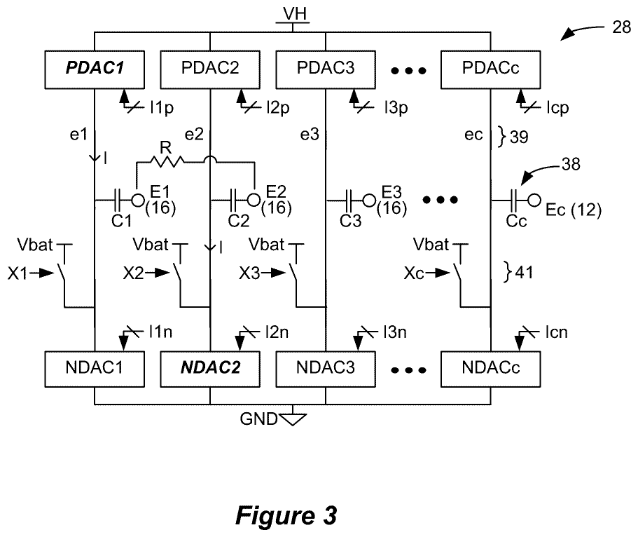

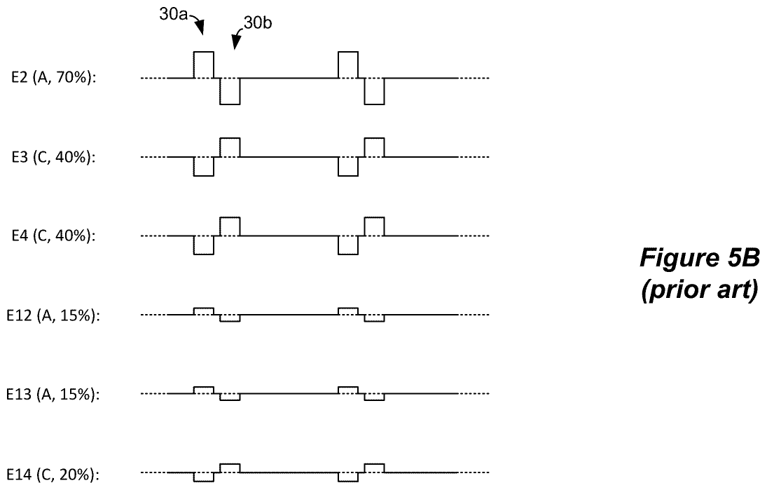

Other stimulation parameters useful when providing SCS to treat conditions impacted by the splanchnic bed include a frequency in the range of 40 to 100 Hz, and a pulse width of 150 to 300 microseconds. Furthermore, it is believed that the provided stimulation should involve the use of active charge recovery—i.e., involve using an active recharge phase 30b following the first phase pulses at 30a (see FIG. 2). Thus, although not shown in FIG. 7, at bipole 1, the polarity would be flipped in this second phase 30b, with electrodes E1-E4 providing cathodic currents, and E5-E8 providing anodic currents. Reversing the polarity at the active electrodes during an active charge recovery phase 30b was described earlier with respect to FIG. 5B. If necessary or desirable, the active charge recovery phase 30b can be followed by the use of passive charge recovery (30c). Although not shown, the selection of the use of active or passive charge recovery can be provided by the GUI 99. Further examples of stimulation parameters useable in this context, and strategies for selecting such parameters, are disclosed in U.S. Patent Application Publication 2020/0009367.

While use of bipolar stimulation (and in particular use of a diffuse bipole) is expected to provide optimal stimulation of the sympathetic splanchnic nervous system, monopolar stimulation may be suitable as well. As discussed above, in monopolar stimulation, the electrode array 17 carries a single anode pole or cathode pole, with the case electrode 12 providing the current return (programmed to provide a pole of the opposite polarity). When using monopolar stimulation, a diffuse pole comprising a plurality of electrodes (e.g., E1-E4, or E5-E8) can be used.

Although the details aren't shown in FIG. 7, the GUI 99 can also be used to schedule the prescribed stimulation. For example, stimulation used to treat conditions impacted by the splanchnic bed can be provided at different locations in the electrode array at different times, and in this regard FIG. 7 shows the use of bipole 1 (E1-E8) and bipole 2 (E9-E16). These bipoles can be interleaved in time, with each being provided for a certain duration (e.g., a minute or so each). In one example, the stimulation provided by different bipoles can be provided in different timing channels in the IPG 10. This stimulation can also be duty cycled, with the stimulation being applied periodically (e.g., ten minutes on), followed by an off period (e.g., ten minutes off). This can be useful in tailoring the dosage of charge or current provided to the patient's tissue.

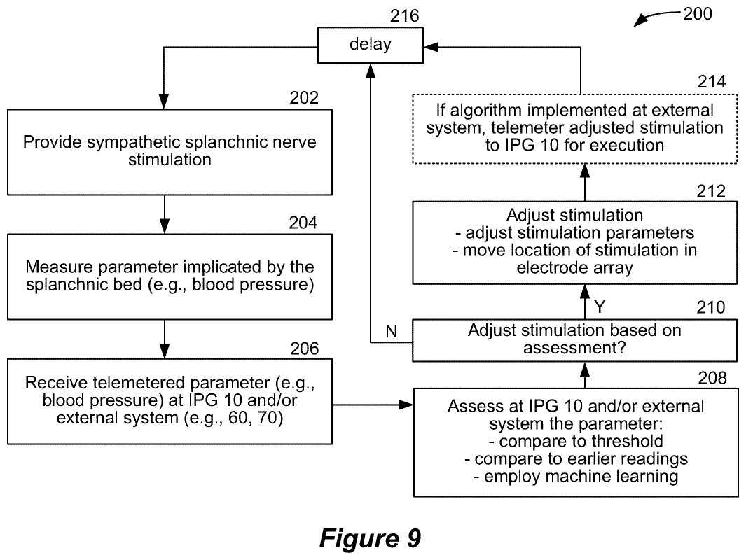

Also included in the GUI 99 of FIG. 7 is a measurement interface 106. This interface 106 allows stimulation to be tailored or adjusted to best treat treating conditions impacted by the splanchnic bed, and to receive inputs regarding various measurements relevant to symptoms. For example, interface 106 includes a selectable option to run a closed loop algorithm 200 that adjusts the stimulation based on various measurements indicative of symptoms. This algorithm 200 is explained further below with reference to FIG. 9. Relevant measurements can be objective or subjective, and can be used to determine if the stimulation is having the intended effect in treating conditions impacted by the splanchnic bed.

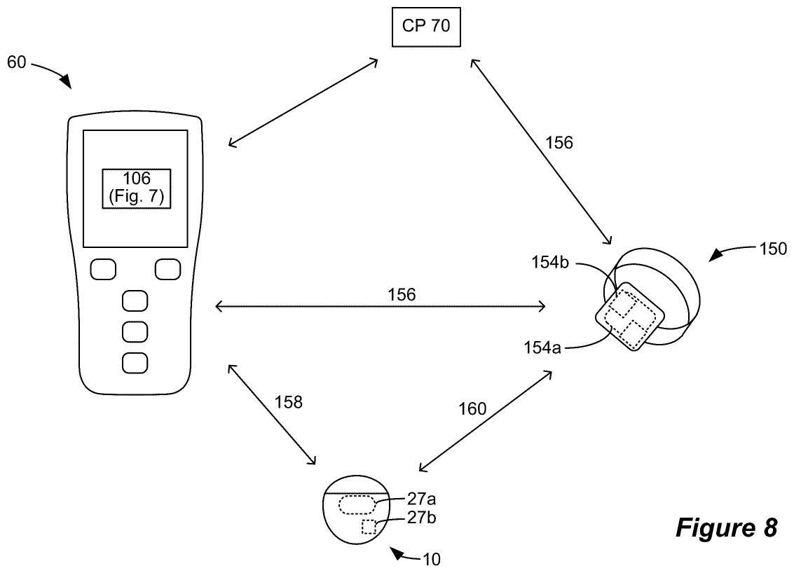

Objective measurements can be input or reviewed at an option 108 in interface 106. As one example of an objective measurement, option 108 shows the blood pressure of the patient. This can be monitored by a sensor 150, as explained further below with reference to FIG. 8. Sensor 150 may automatically wirelessly report measurements to the external system implementing GUI 99. In the example shown, this objective measurement comprises blood pressure, and hence the patient's systolic and diastolic pressures are shown (from 0 to 300 mm Hg). Option 108 may also allow these objective measurements to be manually entered by the clinician, which is useful if the sensor 150 does not automatically report such measurements to the external system.

Option 110 allows subjective measurements to be input in interface 106. Such subjective inputs are not directly and objectively measured, but nevertheless are indicative of a patient's symptoms. Examples of such subjective measurements for a patient predisposed to hearth failure for example include dyspnea (shortness of breath), and walking tolerance, which may be input on a scale of 1 (best) to 5 (worst).

Measurement interface 106 may likewise be present on the patient's external controller 60 (see FIG. 8). This is useful so that the patient's controller may similarly receive or allow the input of subjective or objective measurements relevant to conditions impacted by the splanchnic bed, and to in turn allow the stimulation parameters to be adjusted once initially fitting has occurred using the clinician programmer 70.

As discussed above, one or more sensors 150 can be used to determine if the stimulation is having the intended effect in treating conditions impacted by the splanchnic bed, and an example of a sensor 150 is shown in FIG. 8. The sensor 150 as depicted comprises a cardiac sensor, which can monitor one or more cardiovascular parameters indicative of the effectiveness of the stimulation to modulate blood volume recruitment in the splanchnic bed or more generally to assess the patient's cardiovascular status. In this example, the sensor comprises a blood pressure sensor, which preferably monitors the patient's blood pressure at a peripheral location outside of the splanchnic bed. If stimulation is successful in increasing blood volume in the splanchnic bed for example, the patient's blood pressure would drop when monitored at such a peripheral location. In the example shown, the cardiac sensor 150 is shown as wearable on a patient's wrist or arm, but may take other form factors as well (a patch, a wristwatch, etc.), and may be located at different locations on the patient's body. In other examples, the sensor 150 may be implantable, and thus able to measure the patient's blood pressure internally. Sensor 150 may also be integrated with the IPG 10; the IPG 10 could include a cardiac sensor for example. Sensor 150 may also measure other cardiovascular parameters indicative of the patient's cardiovascular status. Such parameters may include heart rate, heart rate variability, O₂ saturation, various ECG parameters, parameters indicative of vascular resistance, or other cardiovascular parameters.

Sensor 150 may comprise other types of sensors as well, depending on the condition being monitored. For example, sensor 150 could comprise a glucose sensor, a lipid sensor, etc., and may comprise any sensor able to measure a condition impacted by the splanchnic bed.

The sensor 150 is preferably enabled for wireless communications, and may include a near-field magnetic-induction coil antenna 154a and/or a far-field RF antenna 154b. This allows the sensor 150 to communicate its measurements to an external system (such as the clinician programmer 70 or the external controller 60) and/or to the IPG 10 via communication links 156 and 160 respectively. When measurements are communicated with an external system, that system can permit a user to review the measurements, such as by displaying the measurement on a display associated with that system (e.g., in GUI 99). Measurements may also be logged at the external system so that they may be reviewed (e.g., graphed) as a function of time. This is useful to allow a user or clinician to determine if the SCS stimulation is having its intended effect (e.g., lower peripheral blood pressure).

Wireless communication also allows the IPG 10 to be controlled by objective or subjective measurements in a closed loop fashion, thus allowing the IPG 10 to adjust the provided stimulation based upon the measurements. FIG. 9 shows a closed loop algorithm 200 useful in this regard. The algorithm 200 can be implemented within the IPG 10, the external system (e.g., external controller 60, or clinician programmer 70), or both, and variations in this regard are discussed further below. The algorithm 200 may include instructions embodied in one or more non-transitory computer readable media, such as solid state, magnetic, or optical memory, which may reside in the external system (94, FIG. 4), or in the IPG 10. The computer readable medium may also reside in a server in communication with the external system and/or the IPG 10, allowing the algorithm 200 to be downloaded into these devices.

As shown in FIG. 9, the closed loop algorithm 200 starts by providing sympathetic splanchnic nerve stimulation (202), as discussed above. Next, a parameter implicated by the splanchnic bed is measured using (204). As noted earlier, this measurement can be objective (e.g., blood pressure as taken by a sensor 150, and received at option 108 of the GUI, FIG. 7) or subjective (e.g., received at option 110). Next, this measured parameter is received at the external system (e.g., 60, 70), the IPG 10, or both. Receipt of the measurements can occur at device(s) at which the algorithm 200 has been executed, as explained further below. Such receipt may comprise an input received at option 110 if the measurement is subjective, or may be transmitted by telemetry from the sensor 150 if objective.

Next, the received measurement is assessed (208), and such assessment can occur in different manners. In one example, the measured parameter can be compared to a threshold, e.g., a blood pressure threshold that is indicative of a high blood pressure and therefore a low splanchnic bed blood volume. The measured parameter can also be compared to earlier measurements to determine, for example, if the patient's blood pressure is increasing. Machine learning can also be employed to assess the measured parameter. As one skilled in the art will understand, such machine learning can iteratively consider measurements and previous stimulation adjustments (discussed next, step 210) to learn how to control operation of the algorithm for best therapeutic results.

Next, the algorithm 200 determines whether to adjust stimulation based on the assessment of the measured parameter made at step 208 (210). Whether adjusting stimulation is warranted can depend on the particular measurement taken and the manner in which that parameter was assessed. For example, if the measured parameter of blood pressure is high compared to a threshold, or has been increasing, the algorithm 200 may conclude that stimulation adjustment is warranted. As just discussed, machine learning can also determine when it is advisable to adjust the stimulation.

If the algorithm 200 concludes that the stimulation should be adjusted, various manners in which this adjustment can occur are shown in step 212. Adjustment at this step can involve adjustment of any of the stimulation parameters, including amplitude (I), frequency (F), and/or pulse width (PW). Adjustment can also involve adjustment, use, or non-use of various charge recovery periods (30b, 30c) discussed earlier. Such parameter adjustments can be made to one or more of the poles in the electrodes array 17 (e.g., to bipoles 1 and/or 2). The duration at which the poles are applied may also be adjusted, as may the duty cycle of the stimulation. The location of the stimulation in the electrode array 17 can also be varied at this step.

It may not be known in advance how to adjust the parameters or the location of the stimulation. For example, it may not be known whether to increase or decrease amplitude to better affect blood volume recruitment in the splanchnic bed, or whether it would be better to move the location up/down or right/left in the electrode array 17. Nevertheless, because the algorithm 200 will iteratively adjust the stimulation, such adjustments can be made essentially at random to see which adjustments results in therapeutic improvements. For example, the algorithm 200 may decide to increase the amplitude at this step. If this proves to be ineffective based on an assessment of a next-received measurement (206, 208), the algorithm 200 could next try decreasing the amplitude. This is true of other stimulation parameters and the location of the stimulation as well, and essentially the algorithm 200 can operate by randomly or pseudo-randomly adjusting the parameters and/or moving the location of the stimulation until better effectiveness (e.g., lower measured blood pressures) is achieved. Machine learning, if used, can also eventually learn the best manner to adjust the stimulation at step 212.

Step 214 is an optional step that is particularly useful if the algorithm 200 is implemented in an external system (e.g., 60. 70) as opposed to the IPG 10. After the external system has received and assessed the measured parameter (206, 208), and has determined to adjust the stimulation (210, 212), the external system can telemeter the adjusted stimulation to the IPG 10 along communication link 158 (FIG. 8) to be executed. If the algorithm 200 is instead implemented in the IPG 10, with the IPG 10 receiving and assessing the measured parameter (206, 208), and determining on its own to adjust the stimulation (210, 212), this step 214 is unnecessary.

After the simulation is adjusted (212, 214), or if stimulation does not need to be adjusted (210), the algorithm 200 can impart a delay before repeating (216). This ensures that the algorithm 200 does not react too quickly, or make adjustments to the stimulation too frequently. The delay provided at step 216 can generally range from a second to several minutes.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for treating at least one condition impacted by a patient's splanchnic bed using spinal cord stimulation, comprising:
providing an electrode array having a plurality of electrodes within a patient's spinal column; and
providing from a spinal cord stimulator non-destructive electrical stimulation at at least one of the plurality of the electrodes,
wherein the stimulation is configured to affect the sympathetic nervous system innervating the patient's splanchnic bed to modulate one or more of (i) activity of one or more organs in the splanchnic bed, and (ii) a blood volume of the splanchnic bed.

2. The method of claim 1, wherein the stimulation is configured to increase the blood volume of the splanchnic bed.

3. The method of claim 1, wherein the stimulation stimulates one or more dorsal horns of the patient's spinal cord.

4. The method of claim 1, wherein the electrode array is provided longitudinally within the spinal column to configure the stimulation to affect at least one splanchnic nerve innervating the patient's splanchnic bed.

5. The method of claim 1, wherein the stimulation comprises a bipole in the electrode array.

6. The method of claim 1, wherein the stimulation comprises a monopole in the electrode array.

7. The method of claim 1, wherein the stimulation is not perceptible by the patient.

8. The method of claim 1, wherein the electrode array comprises a plurality of electrode leads.

9. The method of claim 1, further comprising measuring a parameter indicative of at least one of the conditions impacted by the patient's splanchnic bed.

10. The method of claim 9, wherein the cardiovascular parameter is indicative of the blood volume of the patient's splanchnic bed.

11. The method of claim 9, wherein the parameter is measured by a sensor.

12. The method of claim 11, wherein the parameter comprises a blood pressure of the patient.

13. The method of claim 9, further comprising adjusting at the spinal cord stimulator the stimulation using the measured parameter.

14. The method of claim 13, wherein the parameter is measured periodically, and wherein the stimulation is adjusted in a closed loop fashion using the periodically measured parameter.

15. The method of claim 9, further comprising wirelessly receiving the measured parameter at the spinal cord stimulator.

16. The method of claim 9, wherein the measured parameter is input using a graphical user interface of an external system in communication with the spinal cord stimulator.

17. The method of claim 9, further comprising wirelessly receiving the measured parameter at an external system in communication with the spinal cord stimulator.

18. A system for treating at least one condition impacted by a patient's splanchnic bed using a spinal cord stimulator comprising an electrode array having a plurality of electrodes configured to be provided in a patient's spinal column, the system comprising:
control circuitry configured to cause non-destructive electrical stimulation at at least one of the plurality of the electrodes, wherein the stimulation is configured to affect the sympathetic nervous system innervating the patient's splanchnic bed to modulate one or more of (i) activity of one or more organs in the splanchnic bed, and (ii) a blood volume of the splanchnic bed;
wherein the control circuitry is further configured to receive a parameter indicative of at least one of the conditions impacted by the patient's splanchnic bed.

19. The system of claim 18, further comprising the spinal cord stimulator, wherein the control circuitry is within the spinal cord stimulator.

20. The system of claim 18, further comprising an external system configured for communication with the spinal cord stimulator, wherein the control circuitry is within the external system.
